(19) United States
(12) Patent Application Publication
(10) Pub. No.: US 2007/0196381 A1
(43) Pub. Date: Aug. 23, 2007

(54) HERBAL COMPOSITIONS, METHODS OF STIMULATING IMMUNOMODULATION AND ENHANCEMENT OF IMMUNOMODULATING AGENTS USING THE HERBAL COMPOSITIONS

(75) Inventor: Stephen Holt, Little Falls, NJ (US)

Correspondence Address: LUCAS & MERCANTI, LLP, 475 PARK AVENUE SOUTH, 15TH FLOOR, NEW YORK, NY, 10016, US

(73) Assignee: NATURES BENEFIT, INC., Little Falls, NJ (US)

(21) Appl. No.: 11/676,082

(22) Filed: Feb. 16, 2007

Related U.S. Application Data
(60) Provisional application No. 60/774,310, filed on Feb. 17, 2006.

(51) Int. Cl. A61K 36/8962; A61K 36/87; A61K 36/09; A61K 36/48; A61K 36/906; A61K 36/886; A61K 36/28; A61K 36/71; A61K 36/06; A61K 36/82; A61K 33/32; A61K 31/375

(52) U.S. Cl. 424/195.15; 424/195.16; 424/725; 424/744; 424/729; 424/756; 424/757; 424/754; 424/726; 424/766; 424/737; 424/641; 514/23; 514/474

(57) ABSTRACT

Herbal based compositions for enhancing immunomodulation in mammals are disclosed in which a combination of herbal products and extracts thereof are included for their complementary and substantially simultaneous effect on different parts of the immune cascade. In preferred aspects, the herbal products and extracts thereof include a mixture of Shiitake Mushroom, Aloe Vera Leaf, Brewer's Yeast, Coriolus Mushroom and AHCC (Active Hexose Correlate Compound). Additional aspects include methods of enhancing immunomodulation and methods of increasing the effectiveness of immunomodulators and/or antiangiogenic administering the inventive compositions either alone or in combination with an immunomodulator or anti-angiogenic compound to a patient in need thereof.

### CROSS-REFERENCE TO RELATED APPLICATION

This application claims the benefit of priority from U.S. Provisional Patent Application Ser. No. 60/774,310 filed Feb. 17, 2006, the contents of which are incorporated herein by reference.

### BACKGROUND OF THE INVENTION

It is known that certain herbal remedies can have a beneficial effect on certain aspects of immune function. See, for example, U.S. Patent Application Publication No. US2006/0009501 which discloses the use of a polysaccharide isolated from Tinospora cordifolia as an immune system stimulator. Similarly, U.S. Patent Application Publication No. US2005/0079234 discloses immunomodulating compositions containing, among other things, American ginseng, licorice root and green tea. The published literature also includes various other herbs which alone or in some combination have a purported effect in supporting immune function and/or boosting the effectiveness of cytokines NK cells etc found in the body.

In spite of the foregoing, there is still a need to provide improved therapies. There is also a need to provide the art with compositions which have a more predictable response in treatment regimens. While Applicant does not wish to be bound by any particular theory, Applicant asserts that the prior art has failed to recognize that more effective immunomodulation and anti-viral therapy could be achieved if the treatments provided a combination of key herbal ingredients which simultaneously support different aspects of the immune system stimulation cascade. This is to be contrasted with previously reported treatments and compositions which targeted specific or random parts of the cascade in efforts to boost immunomodulation.

### SUMMARY OF THE INVENTION

In one aspect of the invention, there are provided herbal based compositions for enhancing immunomodulation in mammals. The compositions include a combination of herbal products and extracts thereof selected for their complementary and substantially simultaneous effect on different parts of the immune cascade. While preferred aspects of the invention will include synergistic combinations of several different herbal products, extracts and ingredients, some especially preferred aspects of the invention include a mixture of at least the following herbal products and extracts of Shiitake Mushroom, Aloe Vera Leaf, Brewer's Yeast, Coriolus Mushroom and AHCC (Active Hexose Correlate Compound). In a separate aspect of the invention, the inventive compositions include not only these four principal ingredients but also effective amounts of Andrographis Paniculata and Eleutherococcus Senticosus, (Siberian Ginseng).

The herbal-based compositions of the present invention are preferably administered in the form of orally-acceptable dosage forms such as a capsule, caplet, compressed tablet or the like. In alternative aspects, the compositions of the invention can be administered as part of any orally acceptable form, including, without limitation, beverages, teas, infusions, etc. The only limitation on the type of dosage form is that the dosage form must not substantially inactivate the immunomodulation enhancement capabilities of the herbal mixtures described herein which are specifically selected for their complementary and substantially simultaneous effect on different parts of the immune cascade.

In a further aspect of the invention, there are provided methods of enhancing immunomodulation in mammals. The methods include administering an effective amount of a composition in accordance with the invention as described herein to a mammal in need thereof. A similar but further aspect of the invention includes methods of increasing or otherwise enhancing the effectiveness of immunomodulators and/or antiangiogenic compositions when administered to patients in need thereof. The methods include administering an effective amount of the inventive compositions described herein in combination with an immunomodulator or anti-angiogenic compound to a patient in need thereof. Such methods include administering the compositions of the present invention in combination with an immunomodulator such as an interleukin, an interferon or other pharmaceutical or biological agent having such activity in mammals, or antiangiogenic compound, such as shark cartilage extract or other pharmaceutical or biological agent having such activity in mammals, to a patient in need thereof.

Certain preferred aspects of the present invention provide methods of increasing natural killer cell activity in mammals. The methods include administering an effective amount of a composition described herein to a mammal in need thereof.

Kits containing the compositions of the present invention and the immunomodulator or antiangiogenic agent are yet a further embodiment of this aspect of the invention.

A further aspect of the present invention provides methods of treatment of diseases caused or effected by malfunction of the immune system. The methods include administering an effective amount of the compositions described herein to a human or animal in need of such treatment.

### BRIEF DESCRIPTION OF THE DRAWING

FIG. 1 shows the results of comparative CD69 induction on human lymphocytes incubated overnight with either MGN-3 or BioDefense extracts as described in Example 6. Untreated cells served as a negative control.

FIG. 2 shows the results of synergy between MGN-3 versus BioDefense extracts with human recombinant IL-2 as in Example 7.

FIG. 3 shows the results of CD25 induction on NK cells after exposure to either MGN-3 or BioDefense extracts as described in Example 8.

### DETAILED DESCRIPTION OF THE PREFERRED EMBODIMENTS

The cascade of immune activation is generally recognized as a complex process in which the body reacts to foreign bacteria, viruses and neoplastic stimuli. Macrophages, T-cells, T-lymphocytes and natural killer or NK cells are all set in motion. The cascade involves the release of cytokines that initiate a series of immunity-related functions, some stimulating the increased formation of white blood cells and white blood cell activity, others sending messages to B and T cells that prompt the adaptive immune response, and others regulating the inflammatory response.

One of the keys to the benefits afforded by the compositions of the present invention is that the mixtures of herbal products and extracts have been shown to have the ability to activate NK cells which can have a beneficial health effect in mammals. Natural killer (NK) cells are a form of cytotoxic lymphocyte which constitutes a major component of the innate immune system and a key part of the immune cascade. NK cells are lymphocytes that originate in the bone marrow and develop fully in the absence of the thymus. Natural killer cells can react against and destroy another cell without prior sensitization to it as part of the body's first line of defense against cancer cells and virus-infected cells. They target tumor cells and protect against a wide variety of infection microbes. Natural killer cells may also contribute to immunoregulation by secreting high levels of influential lymphokines. NK-cells are defined as large granular lymphocytes that do not express T-cell antigen receptors (TCR) or Pan T marker CD3 or surface immunoglobulins (Ig) B cell recptor but which usually express the surface markers CD16 (FcγRIII) and CD56 in humans. Thus, stimulation and/or activation of NK cells in a controlled and predictable fashion would be beneficial.

Oral dosage forms such as capsules or compressed tablets containing the herbal compositions of the present invention can include combinations of the following herbal and natural extracts. It will be appreciated that the artisan will administer 1 or more, preferably about 4, dosage forms containing effective amounts of the ingredients as part of combination daily used to enhance immunomodulation or increase natural killer cell activity, in that aspect of the invention where it is the object to increase said activity. Exact dosages such as the exact amount, frequency and period of administration will vary somewhat according to the needs of the artisan. The compositions described herein can be administered until the desired results are obtained.

Most aspects of the invention include administering the inventive compositions for periods of time which will vary in accordance with the needs of the mammal taking the herbal based compositions. It is further contemplated that the methods of treatment described herein will carry on for as long as needed or desired, if used prophylactically. In those aspects where the compositions of the present invention are administered to enhance or potentiate an immunomodulator or anti-angiogenic agent or pharmaceutical compound, the herbal based compositions can be administered at least as long as the immunodulator or anti-angiogenic compound to the patient in need thereof. The dosing schedule need not be the same as that employed for the immunomodulator or anti-angiogenic compound. Instead the benefits are achieved by concomitant therapy, each according to its known and approved schedule of dosing.

For purposes of the present invention, patients and mammals shall each be understood as including both human and animal subjects who are in need of or who would benefit from the treatments described herein.

For purposes of the present invention, the administration of the compositions of the invention shall be understood to be given “in combination with” the other preferred biologically active agents if they are part of the same course of therapy for a condition or syndrome. Thus, the individual components of the inventive combination need not be given at the exact same time in order for the adjunctive effects to be realized. Instead, the individual components of the combination can be administered in dosing regimens which are separate, if needed, so that maximum therapeutic effectiveness is realized. Furthermore, the individual components need not be given via the same route of administration in order for the increased effectiveness to be observed. Thus, some immunomodulators are given or can be given intravenously while the compositions of the present invention are preferably given by mouth.

For purposes of the present invention, “increasing the effectiveness” of a biologically effective agent such as an immunomodulator or antiangiogenic agent shall be understood to mean that the therapeutic benefit observed by the combination in the methods of treatment is greater than, if not synergistically better than, the therapeutic effect observed when the biologically effective agent is administered alone.

For purposes of the present invention, “immunodulation” shall be understood to mean the ability to effect change in the body's immune system, in either a positive or negative way with regard to health. Agents, whether herbal, biologic or pharmaceutical, which exhibit this effect, i.e. activate or suppress immune system function in mammals, are therefore said to be “immunomodulators”. “Immunostimulants” are those substances that increase the ability of the immune system to fight infection and disease. “Immunoregulation” shall be understood to include various processes by which antibodies may regulate immune responses.

For the purposes of the present invention, the word “pharmaceutical” refers to a material that is a) a synthetically produced bioactive compound, where no structurally identical, naturally produced analog to the synthetically produced bioactive compound exists; or b) a biologically active compound derived from a living organism, where the biologically active compound is not a dietary supplement.

The pharmaceuticals utilized in this invention include the equivalent and alternative salts which may be formulated from the base pharmaceuticals and which achieve substantially the same effect as the pharmaceutical listed.

For the purposes of the present invention, a “dietary supplement” is defined as a product (other than tobacco) that bears or contains one or more of the following dietary ingredients: a vitamin, a mineral, an herb or other botanical, an amino acid, a dietary substance for use by man or other mammal to supplement the diet by increasing the total daily intake of that substance, or a concentrate, metabolite, constituent, extract, or combinations of these ingredients.

The word “nutraceutical,” for the purposes of the present invention, refers to a food item, or a part of a food item, that offers medical health benefits, including prevention and/or treatment of disease. More particularly, a nutraceutical is a material that is: a) a dietary supplement containing a nutritive bioactive compound; or b) a biologically active processed or unprocessed material derived from a plant, a fungus, an animal, or a portion thereof; where the precise composition of the biologically active processed or unprocessed material may be undetermined. Examples of a biologically active processed material may include a finely chopped, powdered, pureed, or cooked material derived from plant or animal tissue, or an extract of plant or animal tissue.

The terms “effective amounts” and “sufficient amounts” for purposes of the present invention shall mean an amount which achieves a desired effect or therapeutic effect as such effect is understood by those of ordinary skill in the art.

As used herein, the term “herbal extract” refers to phytochemicals that are produced in plant tissues and that can be extracted by water, polar, or petroleum solvents, and that have some degree of beneficial health or therapeutic activity. Thus, an herbal extract can be a liquid solution of herbs and alcohol. The dried or fresh herbs can be combined with alcohol, then the solid matter is removed leaving only the oils of the herbs mixed with the alcohol. Processes for obtaining extracts of the herbal products described herein and standard assays concerning the active principles included therein are known to those of ordinary skill. Moreover, herbal extracts of the products described herein are available from commercial sources or are readily obtained without undue experimentation.

In some preferred aspects of the invention the herbal based compositions included in the inventive embodiments are those provided by Natures Benefit, Little Falls, N.J. under the trade name BIODEFENSE™ and Immune Function Naturally™. These products generally contain three groups of compounds: 1) highly purified herbal extracts, 2) vitamin C and zinc, and 3) multiple complex polysaccharides. Without being bound by any theory, it is believed that at least some of the principal the NK activating properties of the compositions described herein is due to the presence a specific array of complex polysaccharides obtained from Shiitake mushroom, Aloe vera (leaf), Coriolus versicolor, AHCC (active hexose correlated compound), and brewers yeast (with its content of beta-glucans). This novel mixture of ingredients has been surprisingly found to uniquely enhance immunomodulation in mammals by acting in concert and act in a way in which each herbal extract or ingredient is complementary with the others and, together have a substantially simultaneous effect on different parts of the immune cascade. This is to be contrasted with some prior art herbal based products which acted on limited parts of the immune cascade and produced uneven or less than desirable results. When these NK-activating multiple complex polysaccharides are combined with other herbal based compositions such as those included in the fully formulated Natures Benefit products, a harmonious and substantially simultaneous stimulation of multiple parts of the immune cascade is observed. The beneficial effects of the herbal based treatment are apparent after a suitable period of initial treatment has been established, i.e. several days or even weeks, so that sufficient levels are attained in the body.

In accordance with the foregoing, some preferred formulations of the present invention contain:

| | Amount per dosage | Amount per dosage |
|---|---|---|
| | Range (mg) | Range (mg) |
| Ingredient | Broad | Preferred |
| Shiitake Mushroom | 5-75 | 15-40 |
| Aloe Vera Leaf | 5-75 | 15-40 |
| Brewer's Yeast | 50-500 | 50-250 |
| Coriolus Mushroom | 0.25-3 | 0.5-1.5 |
| AHCC (Active Hexose | 0.25-3 | 0.5-1.5 |
| Correlate Compound) | | |

More preferably, suitable formulations for use in orally-acceptable dosage forms include:

| | Amount per dosage | Amount per dosage |
|---|---|---|
| | Range (mg) | Range (mg) |
| Ingredient | Broad | Preferred |
| Andrographis Paniculata | 1-50 | 5-15 |
| Eleutherococcus Senticosus | 1-50 | 5-15 |
| (Siberian Ginseng) | | |

Still further suitable orally-acceptable formulations can include:

| | Amount per | |
|---|---|---|
| | dosage | Preferred |
| Ingredient | Range (mg) | (mg) |
| Andrographis Paniculata 95% | 1-50 | 5-15 |
| Eleutherococcus Senticosus 0.8% | 1-50 | 5-15 |
| (Siberian Ginseng) | | |
| Green Tea 50% Polyphenols | 15-100 | 25-75 |
| Turmeric Root | 15-100 | 25-75 |
| Grape Seed 95% | 1-5 | 2-4 |
| Zinc (AAC) | 1-6 | 3-5 |
| Vitamin C | 50-1000 | 100-500 |
| Shiitake Mushroom | 5-75 | 15-40 |
| Echinacea Purpurea Herb | 5-75 | 15-40 |
| Goldenthread Root | 5-75 | 15-40 |
| Aloe Vera Leaf | 5-75 | 15-40 |
| Garlic | 5-75 | 15-40 |
| Astragalus Root | 5-75 | 15-40 |
| Ginseng Panax | 5-75 | 15-40 |
| Brewer's Yeast | 50-500 | 50-250 |
| Coriolus Mushroom | 0.25-3 | 0.5-1.5 |
| AHCC (Active Hexose Correlate | 0.25-3 | 0.5-1.5 |
| Compound) | | |
| Goldenseal Root | 2-20 | 5-15 |
| Ashwagandha | 10-75 | 15-40 |
| Oregon Grape Root | 10-75 | 15-40 |
| pharmaceutical Excipients | qs | qs |

In an alternative embodiment, an orally-acceptable formulation in accordance with the present invention includes

| | Amount per | Amount per |
|---|---|---|
| | dosage | dosage |
| | Range (mg) | Range (mg) |
| Ingredient | Broad | Preferred |
| Vitamin C | 50-1,000 | 100-250 |
| Zinc (Amino Acid Chelate) | 2-50 | 1.5-20 |
| Andrographis Paniculata 10% | 40-1,000 | 60-360 |
| Terpene Lactones | | |
| Green Tea 50% Polyphenols | 25-1,000 | 40-200 |
| Turmeric Root | 25-1,000 | 40-200 |
| Eleutherococcus Senticosus 0.8% | 20-80 | 40-120 |
| Grape Seed Extract 95% OPCs | 1-100 | 2-10 |
| Green Coffee Bean Extract 50% | 5-200 | 10-50 |
| Chlorogenic Acid | | |
| Herbal Blend containing: Ashwagandha | 100-2,500 | 500-1500 |
| Root, Oregon Grape Root, Shitake | | |
| Mushroom, Echinacea Purpurea Herb, | | |
| Goldenthread Root, Aloe Vera Leaf, | | |
| Garlic, Astragalus Root, Ginseng | | |
| Panax, Goldenseal Root, Coriolus | | |
| Mushroom, AHCC (Active Hexose | | |
| Correlate Compound), Saccharomyces | | |
| Spirulina | 2.5-150 | 5-30 |
| Chlorella | 2.5-150 | 5-30 |
| Kelp | 0.1-1.5 | 0.2-0.4 |
| Cayenne Peppers | 1.5-50 | 2.5-15 |

In each case, it will be understood that the formulations will also include all necessary pharmaceutically acceptable excipients needed for making the desired dosage form, i.e. capsule, etc. For purposes of the present invention, the amount for each herbal ingredient mentioned above included in a dosage form shall be understood to be an amount of the herbal ingredient in its commonly obtained form, i.e. dried herb, extract or dried herb extract, etc.

In one preferred embodiment of the invention there are provided methods of enhancing the effectiveness of immunomodulators and/or anti-viral compositions. The methods include the administration of the compositions of the present invention in combination with a known immunomodulator or anti-viral compound to a mammal in need of such treatment. A non-limiting of suitable immunomodulators include interleukins such as IL-1, IL-2, IL-3, IL-4, IL-5, IL-6, IL-7, IL-8, IL-9, IL-10, IL-12 and IL-15 or other known IL's. Preferably the interleukin is IL-2.

The immunomodulator can also be an interferon such as interferon-α, β, or γ. Subtypes of the interferons are also useful. There is no limit to the type of immunomodulator which can be included in the methods of the present invention.

In another aspect of the invention, the antiangiogenesis effect of known agents, including shark cartilage extract, isoflavones and others known to those of ordinary skill in the art, is enhanced by administering the compositions such as BioDefense or others described herein in combination with the antiangiogenic agent to a patient in need thereof.

In each of the foregoing aspects, the amount of the immunomodulator or anti-angiogenic compound administered will be within the same range as that generally accepted by those of ordinary skill. The clinician can modify the amount and/or frequency of the immunomodulator or anti-angiogenic agent after taking into account the effect of the herbal-based compositions described herein without undue experimentation.

In certain preferred aspects, the present invention provides methods of increasing natural killer cell activity in mammals. The methods include administering an effective amount of a composition described herein to a mammal in need thereof. Without being bound by any theory, the compositions described herein substantially increase natural killer cell level in the body.

The methods of this embodiment have utility in the treatment of conditions such as
Cancer: Prostate, breast, colon, and central nervous system cancers
Arthritis: Rheumatoid disease, progressive systemic sclerosis, osteoarthritis, and mixed connective tissue disease.
Skin Conditions: Burns, wound healing, psoriasis, eczema, meangiomas, angiofibroma, Kaposi's sarcoma
Eye Diseases: Diabetic retinopathy, retrolental fibrophasia, mascular degeneration, corneal vascularization, neovascular glaucoma
Inflammatory: Bowel disease, etc.

The simplest way to explain angiogenesis is to consider a four-step process; (1) the localized erosion of the basement membrane in tissues; (2) the migration of activated endothelial cells promoted by angiogenic factors; (3) endthelial cell proliferation; (4) a complex combination of sustaining influences on the angiogenic process. Angiogenic factors and angtiangiogenic compounds may play a role in one or more of these four steps.

The complex steps in the process of angiogenesis and its control provide a multitude of sites for the potential application of antiangiogenic or proangiogenic therapy. The large, ever increasing number of identified angiogenic factors makes it unlikely that one discrete, antiangiogenic molecule can be used as a successful treatment. This reinforces the use of potentially more versatile antiangiogenic agents that may have multiple sites of activity. These agents may be used either alone or in combination with other antiangiogenic compounds of natural origin. Shark cartilage, bovine cartilage, and isoflavones of soyabean origin are suitable antiangiogenic agents for use in the present invention.

### EXAMPLES

The following examples serve to provide further appreciation of the invention but are not meant in any way to restrict the effective scope of the invention.

A selected panel of standard in vitro assays for lymphocyte activation was used on cell cultures of freshly isolated human lymphocytes from healthy donors. Direct NK acitvation was evaluated by immunostaining and flow cytometry for the NK cell activation markers CD69, CD25, and CD54. Proliferation of B, T, and NK cell subsets in vitro was monitored by a flow cytometric method.

### Example 1

### Botanical Extracts

In order to test an herbal based product in accordance with the claimed invention, BioDefense, an herbal based product containing Shiitake Mushroom, Aloe Vera Leaf, Brewer's Yeast, Coriolus Mushroom, AHCC (Active Hexose Correlate Compound) as well as Andrographis Paniculata, Eleutherococcus Senticosus (Siberian Ginseng) and other synergistic ingredients, was obtained from Natures Benefit, Paterson N.J. For comparative purposes, MGN-3, a product from Lane Labs, Allendale N.J. was also tested.

Extracts of each product were prepared in order to conduct in vitro tests. In order to produce extracts of the mainly water-soluble active components in each blend, capsules were opened and one gram of each product added to 10 ml of physiological saline and kept cold and dark for 16 hours. In order to eliminate the large amount of vitamin C from the BioDefense extract, this was subjected to dialysis. Dialysis of MGN-3 did not show any differences and was judged unnecessary. For both extracts, particulate matter was removed by centrifugation, and the supernatant was sterile-filtered using 0.22 nanometer syringe filters. Serial dilutions were prepared in phosphate buffered saline, pH 7.4.

Tests of solubility indicated that the extracted components of BioDefense used in these examples contained 8% of the dry weight of the encapsulated herbal material, whereas the MGN-3 extract used in this study contained 42% of the weight of the encapsulated material.

### Example 2

### Reagents and Monoclonal Antibodies

The human lymphocyte specific monoclonal markers directly conjugated with fluorochromes were purchased from Becton-Dickinson (San Diego Calif.): CD3-PerCP, CD14-PE, CD25-FITC, CD45-FITC, CD54-PE, CD56-FITC, CD56-PE, CD57-FITC and CD69-FITC. Buffers including RPMI-1640, Histopaque, and phosphate-buffered saline were purchased from Sigma-Aldrich (St. Louis, Mo.).

### Example 3

### Purification of Peripheral Blood Mononuclear Cells (PBMC)

Peripheral venous blood samples were obtained, after informed consent, from healthy human volunteers between the ages of 20 and 60 years. Samples were drawn into sodium heparin, and processed within 30 minutes. Whole blood was layered onto Histopaque and centrifuged for 25 minutes of 400 g. The PBMC-rich interface was harvested using a sterile pipette, transferred to sterile vials, and washed twice in phosphate-buffered saline.

### Example 4

### Introduction of Cell Surface Markers

Freshly purified PBMC were resuspended in culture medium, and exposed to serial dilutions of botanical extracts for 18, 24, and 48 hours. Cells were harvested into V-bottom 96-well plates, and washed in phosphate-buffered saline containing 1% bovine serum albumin and 0.02% sodium azide. Cells were resuspended in 50 microliters or buffer, monoclonal antibodies were added in previously established optimal amounts, and incubated in the dark at room temperature for 10 minutes. At additional 110 microliters of buffer was added to each well, and the plates were washed. Cells were resuspended in buffer and transferred to 1% formalin. Samples were stored dark and acquired by flow cytometry within 4 hours. Analysis of fluorescence intensity was performed using the CellQuest software.

### Example 5

### Statistical Analysis

Flow cytometry data was acquired a FacsCalibur flow cytometer and analyzed using the CellQuest Pro software. Further statistical analysis and figures were generated in Microsoft Excel. Statistical significance was tested using Student's t-test with a p value of less than 0.05 indicating a significant difference between treatments.

### Example 6

### Introduction of CD69 Activation Marker on NK Cells

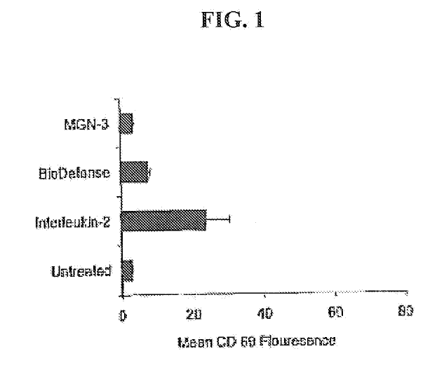

BioDefense treatment of human PBMC in vitro resulted in a robust activation of NK cells, as measured by the induction of the CD69 marker (FIG. 1). MGN-3 treatment did not result in a similar activation, even though some CD69 induction was seen when high doses of MGN-3 extract was used.

Data shown in FIG. 1 are gated for the lymphocyte population using forward and side scatter, and then gated on CD3-negative lymphocytes. Co-staining with CD56-PE and CD69-FITC on these cells showed that among CD56-positive NK cells incubated overnight with MGN-3 only a small subset expressed CD69. In contrast, cells incubated with BioDefense extract showed induction of CD69 on all CD3-negative, CD56-positive NK cells. The data shown are representative for five consecutive experiments.

The effect of both extracts was dose dependant. BioDefense and MGN-3 extracts produced similar levels of NK activation when MGN-3 extract was added at 10-100 fold higher concentration than BioDefense extract. As the water extract of BioDefense contained 5 times less material, based on dry weight estimates, the data indicate at least a 50-fold difference in efficiency between the two extract, with BioDefense being the strongest NK cell activating extract.

### Example 7

### CD69 Induction of BioDefense in Combination with IL-2

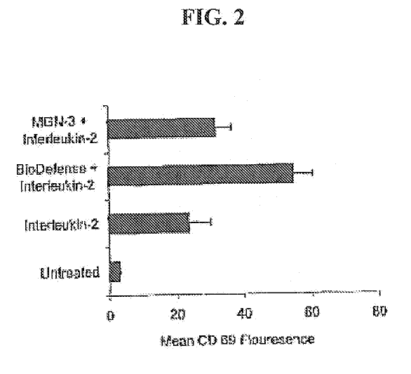

Referring now to FIG. 2, a comparative data prepared as a result of comparing a combination of BioDefense with recombinant IL-2 (r-IL-2), MGN-3 and r-IL-2, IL-2 alone and an untreated control group. Cells were incubated in the presence of extracts with and without addition of IL-2, and the expression levels of CD69 was evaluated by flow cytometry. Histograms show CD69 induction on human CD3-negative, CD56-positive lymphocytes incubated overnight. The data shown are representative for three similar experiments. The data shows that the combination of BioDefense and rIL-2 had more than twice the mean CD69 fluorescence of the rIL-2 alone and a substantially higher amount when compared to BioDefense alone. The testing showed that a previously reported synergy between MGN-3 and IL-2 could not be confirmed with the source of MGN-3 used for these experiments.

### Example 8

### Phenotypic Evaluation of Activated NK Cells

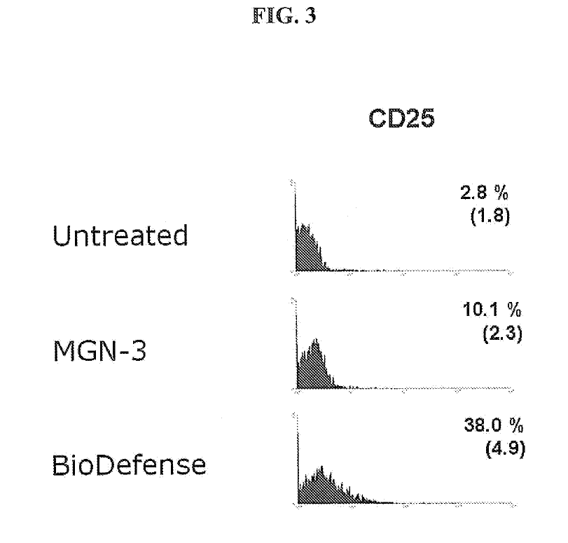

The in vitro activation of NK cells by BioDefense and MGN-3 was analyzed by evaluation of the expression of CD69, CD25 (IL-2 receptor), and CD56. It was found that while CD69 was induced on all cells treated with BioDefense, and a subset of cells treated with MGN-3, only BioDefense was able to significantly induce CD25 on a proportion of the activated NK cells (FIG. 3). BioDefense treatment of the cells also resulted in a slight up-regulation of fluorescence intensity for the CD56 marker on CD3-negative lymphocytes. MGN-3 treatment did not change the expression of CD25 or CD56. None of the extracts changed the expression of CD54 or CD57.

### SUMMARY OF EXAMPLES

Culturing of human lymphocytes in the presence of BioDefense extract resulted in a strong direct activation of NK cells, as evaluated by induction of CD69 on almost 100% peripheral blood NK cells. The effect was concentration-dependant, but substantial NK cell activation was seen over a broad range of dilutions. The data on BioDefense extract show a robust NK activation, as measured by induction of the CD69 marker. MGN-3 produced a weaker, but consistent, activation of NK cells in vitro. Comparison of serial dilutions of both extracts showed that induction of the CD69 NK activation marker required 10-100 fold higher concentrations of MGN-3 extract in order to produce similar activation levels as the BioDefense extract. The combination of BioDefense and rIL-2 induced more than twice the mean CD69 activation of the rIL-2 alone and showed a substantially higher amount when compared to BioDefense alone, MGN-3 alone or in combination with the rIL-2.

I claim:

1. An herbal based composition for enhancing immunomodulation in mammals, comprising a combination of herbal products and extracts thereof selected for their complementary and substantially simultaneous effect on different parts of the immune cascade.

2. The herbal based composition of claim 1, wherein the herbal products and extracts thereof selected for their complementary and substantially simultaneous effect on different parts of the immune cascade comprise a mixture of Shiitake Mushroom, Aloe Vera Leaf, Brewer's Yeast, Coriolus Mushroom and AHCC (Active Hexose Correlate Compound)

3. The herbal based composition of claim 1, further comprising Andrographis Paniculata and Eleutherococcus Senticosus, (Siberian Ginseng).

4. An orally-acceptable dosage form comprising the herbal based composition of claim 1.

5. The orally-acceptable dosage form of claim 4, wherein the herbal products and extracts included therein are present in amounts of:

| | Amount per dosage |
|---|---|
| Ingredient | Range (mg) |
| Shiitake Mushroom | 5-75 |
| Aloe Vera Leaf | 5-75 |
| Brewer's Yeast | 50-500 |
| Coriolus Mushroom | 0.25-3 |
| AHCC (Active Hexose Correlate Compound) | 0.25-3 |

6. The orally-acceptable dosage form of claim 5, further comprising:

| | Amount per dosage |
|---|---|
| Ingredient | Range (mg) |
| Andrographis Paniculata | 1-50 |
| Eleutherococcus Senticosus (Siberian Ginseng) | 1-50 |

7. The orally-acceptable dosage form of claim 5, wherein the herbal products and extracts included therein are present in amounts of:

| | Amount per dosage |
|---|---|
| Ingredient | Range (mg) |
| Shiitake Mushroom | 15-40 |
| Aloe Vera Leaf | 15-40 |
| Brewer's Yeast | 50-250 |
| Coriolus Mushroom | 0.5-1.5 |
| AHCC (Active Hexose Correlate Compound) | 0.5-1.5 |

8. The orally-acceptable dosage form of claim 5, wherein:

| | Amount per dosage |
|---|---|
| Ingredient | Range (mg) |
| Andrographis Paniculata | 5-15 |
| Eleutherococcus Senticosus (Siberian Ginseng) | 5-15 |

9. The orally-acceptable dosage form of claim 5, comprising the formula:

| | Amount per dosage |
|---|---|
| Ingredient | Range (mg) |
| Andrographis Paniculata 95% | 1-50 |
| Eleutherococcus Senticosus 0.8% | 1-50 |
| (Siberian Ginseng) | |
| Green Tea 50% Polyphenols | 15-100 |
| Turmeric Root | 15-100 |
| Grape Seed 95% | 1-5 |
| Zinc (AAC) | 1-6 |
| Vitamin C | 50-1000 |
| Shiitake Mushroom | 5-75 |
| Echinacea Purpurea Herb | 5-75 |
| Goldenthread Root | 5-75 |
| Aloe Vera Leaf | 5-75 |
| Garlic | 5-75 |
| Astragalus Root | 5-75 |
| Ginseng Panax | 5-75 |
| Brewer's Yeast | 50-500 |
| Coriolus Mushroom | 0.25-3 |
| AHCC (Active Hexose Correlate Compound) | 0.25-3 |
| Goldenseal Root | 2-20 |
| Ashwagandha | 10-75 |
| Oregon Grape Root | 10-75 |
| pharmaceutical Excipients | qs |

10. The orally-acceptable dosage form of claim 9, comprising the formula:

| | Amount per dosage |
|---|---|
| Ingredient | Range (mg) |
| Andrographis Paniculata 95% | 5-15 |
| Eleutherococcus Senticosus 0.8% | 5-15 |
| (Siberian Ginseng) | |
| Green Tea 50% Polyphenols | 25-75 |
| Turmeric Root | 25-75 |
| Grape Seed 95% | 2-4 |
| Zinc (AAC) | 3-5 |
| Vitamin C | 100-500 |
| Shiitake Mushroom | 15-40 |
| Echinacea Purpurea Herb | 15-40 |
| Goldenthread Root | 15-40 |
| Aloe Vera Leaf | 15-40 |
| Garlic | 15-40 |
| Astragalus Root | 15-40 |
| Ginseng Panax | 15-40 |
| Brewer's Yeast | 50-250 |
| Coriolus Mushroom | 0.5-0.5 |
| AHCC (Active Hexose Correlate Compound) | 0.5-1.5 |
| Goldenseal Root | 5-15 |
| Ashwagandha | 15-40 |
| Oregon Grape Root | 15-40 |
| pharmaceutical Excipients | qs |

11. The orally-acceptable dosage form of claim 4, comprising the formula:

| | Amount per dosage |
|---|---|
| Ingredient | Range (mg) |
| Vitamin C | 50-1,000 |
| Zinc (Amino Acid Chelate) | 2-50 |
| Andrographis Paniculata 10% Terpene Lactones | 40-1,000 |
| Green Tea 50% Polyphenols | 25-1,000 |
| Turmeric Root | 25-1,000 |
| Eleutherococcus Senticosus 0.8% | 20-80 |
| Grape Seed Extract 95% OPCs | 1-100 |
| Green Coffee Bean Extract 50% Chlorogenic Acid | 5-200 |
| Herbal Blend containing; Ashwagandha Root, | 100-2,500 |
| Oregon Grape Root, Shitake Mushroom, | |
| Echinacea Purpurea Herb, Goldenthread Root, | |
| Aloe Vera Leaf, Garlic, Astragalus Root, | |
| Ginseng Panax, Goldenseal Root, Coriolus | |
| Mushroom, AHCC (Active Hexose Correlate | |
| Compound), Saccharomyces | |
| Spirulina | 2.5-150 |
| Chlorella | 2.5-150 |
| Kelp | 0.1-1.5 |
| Cayenne Peppers | 1.5-50 |

12. The orally-acceptable dosage form of claim 4, comprising the formula:

| | Amount per dosage |
|---|---|
| | Range (mg) |
| Ingredient | Preferred |
| Vitamin C | 100-250 |
| Zinc (Amino Acid Chelate) | 1.5-20 |
| Andrographis Paniculata 10% Terpene Lactones | 60-360 |
| Green Tea 50% Polyphenols | 40-200 |
| Turmeric Root | 40-200 |
| Eleutherococcus Senticosus 0.8% | 40-120 |
| Grape Seed Extract 95% OPCs | 2-10 |
| Green Coffee Bean Extract 50% Chlorogenic Acid | 10-50 |
| Herbal Blend containing: Ashwagandha Root, | 500-1500 |
| Oregon Grape Root, Shitake Mushroom, | |
| Echinacea Purpurea Herb, Goldenthread Root, | |
| Aloe Vera Leaf, Garlic, Astragalus Root, | |
| Ginseng Panax, Goldenseal Root, Coriolus | |
| Mushroom, AHCC (Active Hexose Correlate | |
| Compound), Saccharomyces | |
| Spirulina | 5-30 |
| Chlorella | 5-30 |
| Kelp | 0.2-0.4 |
| Cayenne Peppers | 2.5-15 |

13. A method of enhancing immunomodulation in mammals, comprising administering an effective amount of a composition of claim 1 to a mammal in need thereof.

14. A method of enhancing immunomodulation in mammals, comprising administering an effective amount of an oral dosage form of claim 4 one to four time daily to a mammal in need thereof.

15. A method of increasing the effectiveness of immunomodulators and/or antiangiogenic compositions when administered to patients in need thereof, comprising administering a composition of claim 1 in combination with an immunomodulator or anti-angiogenic compound to a patient in need thereof.

16. The method of claim 15, wherein the immunomodulator is an interleukin.

17. The method of claim 16, wherein the interleukin is selected from the group consisting of IL-1, IL-2, IL-3, IL-4, IL-5, IL-6, IL-7, IL-8, IL-9, IL-10, IL-11, IL-12 and IL-15.

18. The method of claim 17, wherein the interleukin is IL-2.

19. The method of claim 15, wherein the immunomodulator is an interferon.

20. The method of claim 19 wherein the interferon is selected from the group consisting of interferon-α, β, and γ.

21. The method of claim 15, wherein the antiangiogenic agent is shark cartilage extract or an isoflavone.

22. A method of increasing natural killer cell activity in mammals, comprising administering an effective amount of a composition of claim 1 to a mammal in need thereof.

23. A kit comprising an effective amount of the composition of claim 1 and an effective amount of an immunomodulator or antiangiogenic agent for use in the enhancement of immunomodulation in patients.
